(19) United States
(12) Patent Application Publication
(10) Pub. No.: US 2011/0224625 A1
(43) Pub. Date: Sep. 15, 2011

(54) Device for Intraluminal Drug Delivery

(76) Inventors: William J. Flickinger, Lino Lake, MN (US); Steven F. Isenberg, Fishers, IN (US)

(21) Appl. No.: 13/046,350

(22) Filed: Mar. 11, 2011

Related U.S. Application Data
(60) Provisional application No. 61/312,942, filed on Mar. 11, 2010.

(51) Int. Cl. A61M 5/14

(52) U.S. Cl. 604/257

(57) ABSTRACT

The present invention provides a method and device for irrigation and topical delivery of materials within body lumens accessible by natural orifices. The device includes a catheter body containing a delivery lumen for transporting material and is pre-shapeable at room temperature. A connector such as a luer lock connector is located at the proximal end of the catheter body for connecting the device to a reservoir, and a tip with axial and/or radial exit holes is located at the distal end of the catheter body for insertion into body lumen. The tip is larger in diameter than the catheter body, thereby diffusing insertion pressure across a wider area, thus minimizing abrasion, contusion and accidental puncture of body tissue. The proximal end of the tip is tapered to the diameter of the catheter body to allow withdrawal of the tip without causing tissue trauma.

### CROSS-REFERENCE TO RELATED APPLICATIONS

This application claims the benefit of and priority to U.S. Provisional Patent Application No. 61/312,942 filed Mar. 11, 2010 the technical disclosures of which are hereby incorporated herein by reference.

### TECHNICAL FIELD

The present invention relates to the delivery of fluid, powder, gas or gel to a lumen within the body for irrigation, delivery of biologically active materials or for the delivery of medication.

### BACKGROUND OF THE INVENTION

Catheters and other devices such as trochars intended to deliver irrigants, medication or biologicals within the body have conflicting requirements of pushability to deliver the tip of the catheter or device while at the same time not causing inadvertent damage to tissue, including but not limited to punctures, abrasions or contusion to structures in or near the target area.

Inadvertent puncture of the mucosal lining of the body lumen can result in the inadvertent delivery of materials to areas of the body other than the target areas, which can result in significant harm or even death to the patient. For example, intranasal delivery of drugs intended for mucosal delivery that are in advertently delivered into the sub-mucosal space may occlude the ocular veins, which can result in blindness. As another example, materials delivered to the lower esophageal sphincter region which are inadvertently delivered across the esophagus and into the thoracic or abdominal space have been shown to migrate to the aorta and cause rupture and subsequent death.

Soft devices require guide wires or guide catheters to stiffen them, and devices that are stiff enough to advance into the lumen without such guide wires or catheters may be too stiff and cause inadvertent damage as described above. Therefore, it would be desirable to have a method and device for the topical delivery of materials to lumenal structures in the body, specifically structures accessed by open orifices including the nose, mouth, ear urethra and anus, that does not suffer from the risks of inadvertent injury associated with prior art devices.

### SUMMARY OF THE INVENTION

The present invention provides a method and device for irrigation and topical delivery of materials within body lumens accessible by natural orifices. The device includes a catheter body containing a delivery lumen for transporting material. The catheter body is pre-shapeable at room temperature and holds its shape during insertion into body lumen. In one embodiment, the catheter body also includes a wire lumen that runs parallel to the delivery lumen and holds a malleable wire used for pre-shaping the catheter body before use. A connector such as a luer lock connector is located at the proximal end of the catheter body for connecting the device to a reservoir, and a tip is located at the distal end of the catheter body for insertion into body lumen. The tip includes axial and/or radial exit holes for delivery of therapeutic material transported through the catheter body. The tip is larger in diameter than the catheter body, thereby diffusing insertion pressure across a wider area, thus minimizing abrasion, contusion and accidental puncture of body tissue. The proximal end of the tip is tapered to the diameter of the catheter body to allow withdrawal of the tip without causing tissue trauma.

The tip may have a durometer that is softer than target tissue in the body lumen, causing the device to deflect away from the tissue when it makes contact, thereby preventing tissue damage and is made from biocompatible materials such as, e.g., polycarbonate, PVC, silicone, thermoplastic polyurethane (TPU) and thermoplastic elastomers (TPE) such as PEBAX®. The tip can be a molded tip that is attached to the distal end of the catheter body or made from an extension of the catheter body material.

### BRIEF DESCRIPTION OF THE DRAWINGS

The novel features believed characteristic of the invention are set forth in the appended claims. The invention itself, however, as well as mode of use and advantages thereof, will best be understood by reference to the following detailed description of illustrative embodiments when read in conjunction with the accompanying drawings, wherein:

FIG. 1 shows one embodiment of the device with a tip that is larger in diameter than the catheter body in accordance with the present invention;

FIG. 2 shows a detailed view of the catheter tip in accordance with the present invention;

FIG. 3 shows a cross section view of the device with a detailed cross section view of the tip in accordance with the present invention;

FIG. 4 shows an embodiment of the body section of the device with a delivery lumen and a lumen containing a wire for shaping the device in accordance with the present invention;

FIG. 5 shows the delivery device wherein the tip is formed from an extension of the catheter body material in accordance with an embodiment of the present invention; and

FIG. 6 shows another embodiment of the delivery device wherein the catheter body has a pleated portion that enables the body to be formed and hold its shape.

### DETAILED DESCRIPTION OF THE DRAWINGS

The present invention improves upon current delivery systems and provides a method of delivering materials directly to bodily lumens reached by natural orifices while protecting the soft tissue found in these lumens from inadvertent abrasion, contusion or puncture. Examples of targets areas of treatment include the nasal cavity and sinuses, the digestive syste, the urinary system, the ear canal, the lower respiratory system, the lacrimal canal, and the Eustachian tube.

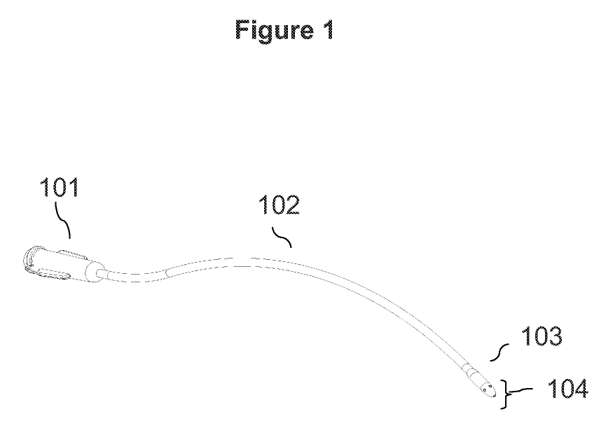

FIG. 1 shows an embodiment of the delivery device in accordance with the present invention. In the example shown, the delivery system has a luer lock connector 101 at the proximal end that can be attached to a standard syringe or other reservoir. The luer lock connector 101 is attached to a catheter body 102 that is in turn attached to a distal tip 103. The body 102 is stiff and formable in order that the device may be pre-shaped by the user for insertion into a body lumen and retain is shape during use.

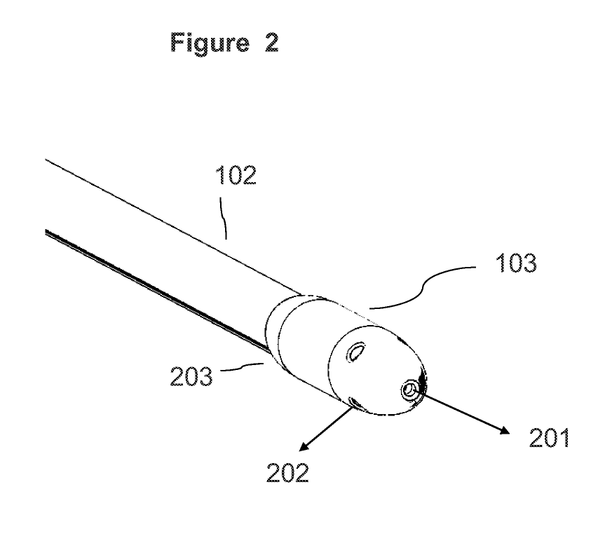

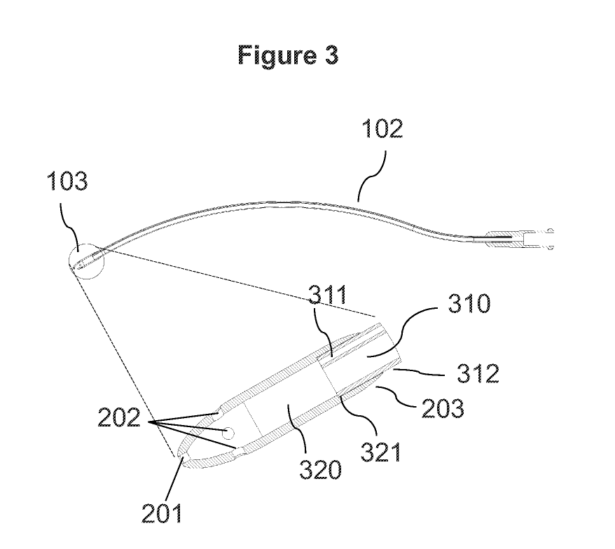

The tip 103 includes exit holes 104 (shown in more detail in FIGS. 2 and 3) through which material can be delivered to the target tissue. The tip 103 is larger in diameter than the body 102 in order to diffuse the force of insertion across a wider area of tissue, thereby minimizing abrasion, contusion or accidental puncture of the tissues in the target area which could lead to unintended sub-topical delivery of material.

FIG. 2 shows a detailed view of the catheter tip in accordance with the present invention. In the embodiment shown the tip 103 is larger (wider) than the body 102 of the device. As shown in the figure, the tip 103 includes an axial hole 201 and several radial holes 202 through which material such as medication, saline solution, etc., may be delivered to the target tissues from the catheter.

The larger surface of the tip 103 diffuses the insertion pressure across a wider area of tissue and is shaped to float on the surface of the body lumen. A taper section 203 at the proximal end of the tip 103 provides a smooth transition between the narrower diameter of the catheter body 102 and the larger diameter of the tip 103 (shown more clearly in FIG. 3), thereby allowing for atraumatic withdrawal of the catheter.

FIG. 3 shows a cross section view of the device with a detailed cross section view of the tip in accordance with the present invention. This view shows in more detail how the body 102 of the catheter mates with tip 103. As shown in the figure, the delivery lumen 310 of the body 102 empties into the lumen 320 of the tip 103. The tip lumen 320 has a slightly larger diameter than that of the delivery lumen 310. The exit holes 201, 202 in the tip 103 in turn are smaller than either lumen 310, 320.

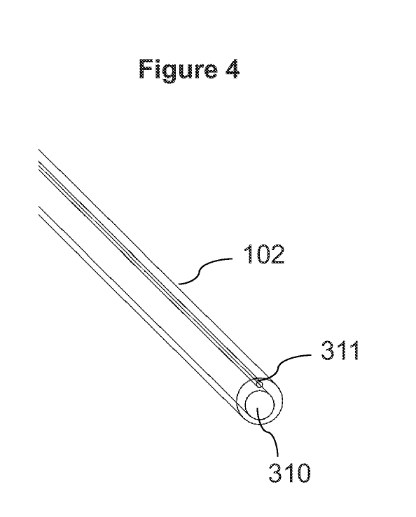

This configuration enables the tip lumen 320 to act as a manifold that equalizes the pressure of the material to be delivered so that it flows out of each exit hole 201, 202 at an equal rate, all other things being equal. The wire lumen 311 (see FIG. 4) opens into the tip lumen 320 as well to allow the wire to move into and out of the tip lumen as the wire is shaped so that the wire does not force the tip 103 to accidentally come off the body 102 of the device.

The taper section 203 at the proximal shoulder of the tip mates smoothly with the outside surface 312 of the catheter body 102 to allow atraumatic withdrawal of the tip 103, as explained above. The tip 103 also features an inner stop 321 that stops the catheter body 102 from infringing too deeply into the tip lumen 320.

As explained above, the tip of the device can be a molded tip that is attached to the body. The tip can be a softer durometer tip that is softer than the target tissue in the body lumen so that the tissue deflects the device when it comes into contact, thereby preventing inadvertent abrasion, contusion or puncture. This softer durometer tip can be isodiametric with the body so that the device is easily compatible with guide catheters, endoscopes or other introducers. The tip can be made from a variety of biocompatible materials including, e.g., polycarbonate, PVC, silicone, thermoplastic polyurethane (TPU) and thermoplastic elastomers (TPE) such as PEBAX®.

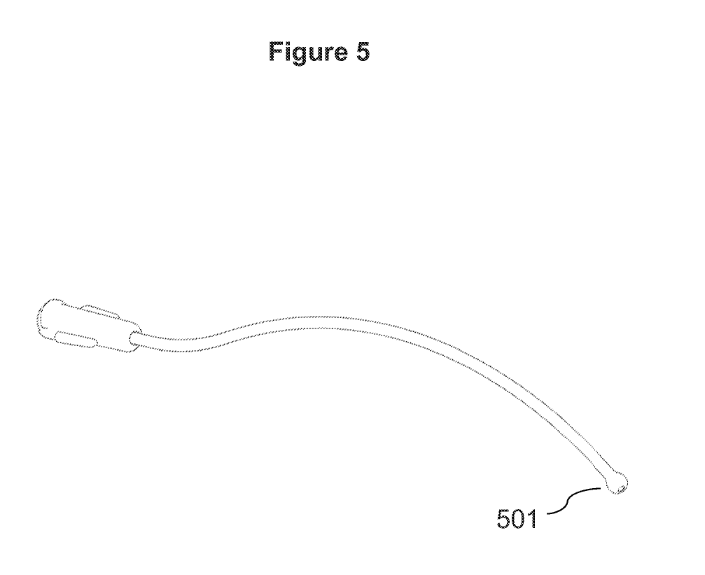

Alternatively, the tip of the delivery device can be a formed tip made from an extension of the body material. This embodiment is shown in FIG. 5. The bulbous formed tip 501 has a large surface area that diffuses the insertion pressure across a wide area of tissue and is shaped to float on the surface of the lumen.

FIG. 4 shows an embodiment of the body section of the device with a delivery lumen and a lumen containing a wire for shaping the device in accordance with the present invention. In this embodiment, the catheter body 102 includes a material delivery lumen 310 and a wire lumen 311 that holds a malleable wire (not shown) that enables the user to pre-shape the device prior to insertion. The wire allows the body to hold this shape during use. The catheter body can be made from a variety of biocompatible materials such as polycarbonate, PVC, silicone, TPU and TPE.

In another embodiment, the body is a simple tube with adequate stiffness. The tube is made of a suitable polymer that can be shaped by the user at room temperature and hold its shape.

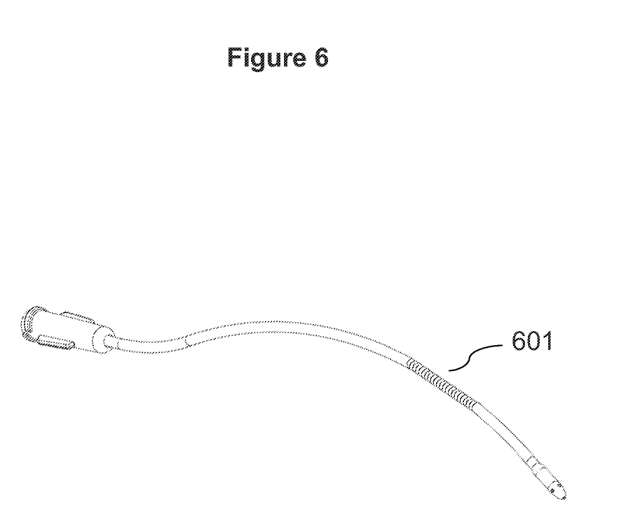

In yet another embodiment, shown in FIG. 6, the catheter body is a thin-walled polymer tube with at least a portion 601 having pleating that enables the body to be formed and hold its shape, analogous to a drinking straw.

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated. It will be understood by one of ordinary skill in the art that numerous variations will be possible to the disclosed embodiments without going outside the scope of the invention as disclosed in the claims.

We claim:

1. A device for intralumenal irrigation and delivery of therapeutic materials, the device comprising:
(a) a catheter body containing a delivery lumen for transporting material, the catheter body including a proximal and a distal end, wherein the catheter body is pre-shapeable at room temperature and holds its shape during insertion into body lumen;
(b) a connector at the proximal end of the catheter body for connecting the device to a reservoir;
(b) a tip at the distal end of the catheter body for insertion into body lumen, wherein the tip is larger in diameter than the catheter body, thereby diffusing insertion pressure across a wider area than the catheter, thus minimizing abrasion, contusion and accidental puncture of body tissue, and wherein the tip includes at least one exit hole for delivery of material transported through the catheter body.

2. The device according to claim 1, wherein the connector at the proximal end is a luer lock connector.

3. The device according to claim 1, wherein the tip has a proximal end that is tapered to the diameter of the catheter body to withdrawal of the tip without causing tissue trauma.

4. The device according to claim 1, wherein the tip includes an axial exit hole for the delivery of therapeutic material to target tissue.

5. The device according to claim 1, wherein the tip includes at least multiple radial exit holes for the delivery of therapeutic material to target tissue.

6. The device according to claim 1, wherein the tip includes an axial exit hole and multiple radial exit wholes for the delivery of therapeutic material to target tissue.

7. The device according to claim 1, wherein the tip is isodiametric with the catheter body.

8. The device according to claim 1, wherein the tip has a lumen that is larger in diameter than the exit holes and delivery lumen of the catheter body, thereby causing the lumen in the tip to act as a manifold that equalizes pressure and causes therapeutic material to flow out of the exit holes at an equal rate.

9. The device according to claim 1, wherein the tip has a durometer that is softer than target tissue in the body lumen, causing the device to deflect away from the tissue when it makes contact, thereby preventing tissue damage.

10. The device according to claim 9, wherein the tip is made from at least one of the following materials:
Polycarbonate;
PVC;
Silicone;
thermoplastic polyurethane;
thermoplastic elastomers.

11. The device according to claim 1, wherein the catheter body is made from at least one of the following materials:
Polycarbonate;
PVC;
Silicone;
thermoplastic polyurethane;
thermoplastic elastomers.

12. The device according to claim 1, wherein the catheter body further comprises an wire lumen that runs parallel to the delivery lumen and contains a malleable wire used for pre-shaping the catheter body before use.

13. The device according to claim 1, wherein the catheter body has at least a portion having pleating that enables the catheter body to be pre-shaped.

14. The device according to claim 1, where the tip is molded tip that is attached to the distal end of the catheter body.

15. The device according to claim 1, wherein the tip is a formed tip made from an extension of the catheter body material.
